(19) United States
(12) Patent Application Publication
(10) Pub. No.: US 2008/0003190 A1
(43) Pub. Date: Jan. 3, 2008

(54) Antibacterial botanicals

(76) Inventor: John Jacob Wille JR., Chesterfield, NJ (US)

Correspondence Address: MILDE & HOFFBERG, LLP, 10 BANK STREET, SUITE 460, WHITE PLAINS, NY, 10606, US

(21) Appl. No.: 11/821,288

(22) Filed: Jun. 22, 2007

Related U.S. Application Data
(60) Provisional application No. 60/817,918, filed on Jul. 3, 2006.

(51) Int. Cl. A61K 8/97; A61K 36/53; A61P 31/00; C12Q 1/02; C12Q 1/18; A61Q 15/00; A61K 36/82

(52) U.S. Cl. 424/65; 424/729; 424/745; 424/774; 424/776; 424/778; 435/29; 435/32

(57) ABSTRACT

A method is disclosed for the preparation of an antimicrobial botanical active from a natural plant. The method comprises the steps of:
(a) selecting a plant from the group consisting of: Allspice (Pimenta diocia), Applephenon®, a commercially available green apple extract (Nikka Whisky Distillery, Ashai Breweries Ltd, Tokyo, JP), ripe autumn olive berry (Eleagnus umbelifera), dried “Baes pepper korns from the Island of Guadalupe, commercially available cinnamon powder, commercially available cocoa powder, cloves (Syzgium aromaticum), corn tassels (Zea mays), curly dock seeds (Rumex crispus), dried hops leaves (Humulus lupulus), dried Lavender flowers (Lavendula provence), Linden tree catkin (Tilia americana), dried Oak tree catkins (Quercus alba), dried Oregano leaves (Origanum vulgare), chinese Prickly Ash (Pericarpium zanthoxylli), and commercially available dried green tea leaves (Camellia sinensis); and (b) forming a natural botanical extract from said plant in an amount effective for exerting at least one of the following properties and physiological actions on microbes: (1) inhibiting the growth of gram-positive and gram-negative microorganisms, and (2) inhibiting the growth of yeast and fungi.

### CROSS-REFERENCE TO RELATED APPLICATION

This application claims priority from provisional Application 60/817,918 filed Jun. 30, 2006, entitled “Antibacterial Botanicals.”

### FIELD OF THE INVENTION

The field of the invention is in cosmetic, wound care, and personal care formulations that employ natural botanical ingredients with anti-microbial activity.

### BACKGROUND OF THE INVENTION

Staphylococcus epidermis is a common bacterium associated with malodor in human underarms. Antiperspirant (AP) and anti-odor (AO) personal care products generally contain antibacterial agents to eliminate odors. Recent interest has shifted to the use of safe natural ingredients to kill malodorous bacteria in AP/AO products.

Previous patents have disclosed the antimicrobial action of essential oils, e.g., limonine, geraniol, and other terpenoids , e.g., Chastain D E et al, U.S. Pat. No. 5,308,873, 1994; “Method for killing yeast and fungi with Caveol”, as well as for treating inflammatory skin disorders (Gendimenico G J, U.S. Pat. No. 6,881,756, 2005). Zhou J H in U.S. Pat. No. 6,139,523, (2001) disclosed the use of polyphenolic derivative composition from licorice extracts combined with a mongroside to inhibit the growth of oral microbials. Iwai K in U.S. Pat. No. 6,387,417 (2002) discloses the use of metal complexes with the essential oil Hinokitiol as a bactericide against vancomycin-resistant enterococcus bacteria, and Hamilton-Miller J M C in U.S. Pat. No. 5,879,683 (1999) describes a method for inhibiting the production of bacteria which constitutively express PBP2 in combination with the antibiotic beta-lactam using tea extracts that is active against methacillin-resistant S. aureus (MRSA). Among the actives in tea extracts with antibacterial activity are catechins e.g., trihydroxybenzoic acid. Finally, Aga H et al in U.S. Pat. No. 6,524,625 (2003) describe the isolation of antibacterial principles from than indigo plant. Among the active agents reported are Kaemerferol, gallic acid, caffeic acid, indirubin, pheophenorbide and trptanrthrin. In U.S. Pat. No. 5,994,413 Tanabe et al., (1999) disclose methods for the preparation of an apple extract enriched in polyphenols and in a special issue of BioFactors volume 22, numbers 1-4(2004) Yoko Akazone reported that apples contain many kinds of antioxidant polyphenols, the main ones being oligomeric procyanidins. A commercial preparation of this apple extract, Applephenon(&, was produced from unripe apples, and has been used as a food additive.

### SUMMARY OF THE INVENTION

The present invention concerns a method for the extraction of botanical materials that possess both antimicrobial and antioxidant activity. A principal objective of the invention is to reveal the presence of antimicrobial activity derived from both wild plants and a variety of commonly cultivated herbs, spices and medicinal plants.

This objective, as well as further objectives which will become apparent from the discussion that follows, are achieved, in accordance with the present invention, by providing methods for the successful preparation, as well as the utilization of antibacterial activity, of many different botanical extracts that are effective antibacterial agents against both the gram-positive bacterium, Staphylococcus epidermis, isolated from human armpit skin, yeast cells (Saccharomyces cerevisae), Pseudomonas aeruginosa, and gram-negative bacteria also isolated from underarm skin isolates. In practice, the botanical extracts are prepared as water or alcohol extracts, which are diluted into saline solutions to ascertain their antibacterial activity.

The microbiological methods and results obtained in accordance with the invention confirm that all of the following examples of botanical extracts have antibacterial activity against underarm derived S. epidermis: Allspice (Pimenta diocia), Applephenon®, a commercially available green apple extract (Nikka Whisky Distillery, Ahi BreweriesLtd, Tokyo, JP), ripe autumn olive berry (Eleagnus umbelifera), dried “Baes pepper korns from Guadalupe, commercially available cinnamon powder, commercially available cocoa powder, cloves (Syzgium aromaticum), corn tassels (Zea mays), curly dock seeds (Rumex crispus), dried hops leaves (Humulus lupulus), dried Lavender flowers (Lavendula provence), Linden tree catkin (Tilia Americana), dried oak tree catkin (Quercus alba), dried Oregano leaves (Origanum vulgare), Chinese Prickly Ash (Pericarpium zanthoxylli), and commercially available dried green tea leaves (Camellia sinensis). Several botanical extracts including Citricidal®, a commercial grape seed extract, and HIDROX®, a commercial water-soluble extract of green olive pulp, and White Willow Bark extract were included as positive controls in the microbiological assays as they are known antioxidants and antimicrobial agents.

A further objective of the present invention is to confirm the purported fact that the demonstrated bactericidal activity of all of the cited botanical extracts has a strong correlation to the polyphenolic content of the extracts. This surmise was supported by their positive Ferric Chloride reactivity, assayed calorimetrically by an instantaneous color change from an orange straw color of the un-reacted aqueous Ferric Chloride solution (10%) to a green-black color.

### BRIEF DESCRIPTION OF THE DRAWINGS

FIG. 1 is a photograph of a 96-well culture dish showing the Ferric Chloride (FeCl₃) reaction with various test botanical extracts, Detection of polyphenols gives a black color. Light orange color is of no reaction as described in text. Table 1 lists all of the botanicals tested in the ferric chloride assay.

FIG. 2 is a photograph of the results of a self-sterility result (negative, −) of powdered corn tassel extract (here designated Tasselin). The powdered corn tassel extract was extracted sequentially with distilled water (A), 100% methanol (B), and 70% ethanol. (C), with 10 microliters (AL) of each inoculated into 2 ml of sterile Beef Heart Infusion (BHI) bacterial growth medium.

FIG. 3 is a photograph showing the bactericidal activity of a 20% (⅕×) of full strength Autumn Olive ripe berry extract. Note: the left and right dishes are duplicate dishes. Negative (−) means no bacterial growth. Positive (+) means bacterial growth.

FIG. 4 is a photograph showing the bactericidal activity of a 1% (w/v) water-soluble extract of powdered ApplephenonSH® (top right) and a 1% (w/v) water extract of HIDROX® (top middle). The positive control (+), i.e., inoculated with bacteria, is shown in the top left. The word “empty” denotes the absence of any test in that well.

FIG. 5 is a photograph showing the bactericidal activity of a 0.5% of full strength extract (10% w/v) of dried green tea leaves. Saline controls without green tea extract contained 0, 5% and 10% alcohol) and the green tea extracts contained only 5% and 10% alcohol. All of the controls were heavily contaminated with S. epidermis. The 6-well dish designated “Blank” is an empty well.

FIG. 6 is a photograph showing the bactericidal activity (bottom left clear well) of a 1% (w/v) water-soluble extract of dried Prickly Ash (P.z., Pericarpium zanthoxylli) extract. The saline positive control (top right dark well) was spiked with a culture of S. epidermis). The word “empty” denotes the absence of any test in that well.

FIG. 7 is a photograph of duplicate dishes showing the bactericidal activity (Negative) of a water-soluble extract (0.4%) of powdered corn tassel extract spiked with a culture of S. epidermis (bottom 2 clear wells). Positive control spiked with S. epidermis (top row of duplicate dark-colored wells) showing strong bacterial growth without Tassel powder yet spiked with S. epidermis bacteria.

FIG. 8 is a photograph showing the bactericidal action of 1% of a 95% ethanol extract of corn tassels (Tasselin) against Pseudomonas aeruginosa (top left well-containing 10% alcohol). Positive saline control (dark colored left bottom well-10% alcohol) without corn tassel (Tasselin) showed heavy P. aeruinosa bacterial growth.

FIG. 9 is a photograph showing the bactericidal activity of a 2% (⅕×) and 1% (1/10×) of extracts of dried Pimenta seeds (Jamaica, Ja), with no ethanol (bottom row, water extract), and with ethanol (top row, 10% alcohol extract, Alc). Alcohol control (top dark left well) and water control (bottom dark left well) were heavy growth of S. epidermis. Note: 1% alcohol Pimenta extract was also contaminated (top dark right well).

FIG. 10 is a photograph showing the bactericidal activity of three different botanicals at 20% (⅕×) of full strength hydro-alcoholic extracts of Oregano leaves (Bottom left), dried Oak tree catkin (bottom middle), and dried Hops flowers (bottom right). The positive control (+) is shown in the top right well. The word “empty” denotes the absence of any test in that well.

FIG. 11 is a photograph showing the bactericidal activity of three different botanicals at 20% (⅕×) of full strength hydroalcoholic extracts of dried Curly Dock seeds (bottom left), Lavander flowers (bottom middle), and dried Baes “pepper korns” from Guadalupe (bottom right). The positive control (+) is shown in the top right well. The word “empty” denotes the absence of any test in that well.

FIG. 12 is a photograph showing the bactericidal activity of ApplephenonSH at 1%, 0.5% and 0.25%. The minimal bactericidal concentration of ApplephenonSH was determined to be below 0.063%. The positive control (+) is shown in top left well. Bacteria were seen in the well by microscopic examination. The word “empty denote the absence of any tests in those wells.

FIG. 13 is a photograph showing bactericidal activity is present in ApplephenonSH® extracts prepared either in water at pH 3.5 or at pH 7.0. The neutralized AP extract turns red (dark color), while the 1% AP at pH 3 was yellow (light color). Both were free of bacterial contamination, after challenge with S. epidermis.

FIG. 14 is a photograph showing the minimal bactericidal concentration of water soluble extract of ApplephenonSH® (top two left wells) is determined to be about 0.125% against the gram-negative bacterium, Pseudomonas aeruginosa. Both the AP extract at 0.063% (top right well) and the saline control (bottom left well), were heavily contaminated with P. aeruginosa bacteria.

FIG. 15 is a photograph showing the bactericidal activity of whole cloves (WC) and Anise at 20% (⅕×) of full-strength of extracts prepared in water (H2O) or 50% ethanol (Etoh). The word “empty” denotes the absence of any tests in those wells. The saline control (bottom left well), and the 20% (⅕×) Anise-H 2O both spiked with S. epidermis bacteria were heavily contaminated.

FIG. 16 is a photograph showing the bactericidal activity of a pair-wise combination of ApplephenonSH® and Green Tea extract (AP+GT, 0.03%) versus ApplephenonSH® (AP, 0.03%) only and Green Tea extract (GT, 0.03%) only.

### DETAILED DESCRIPTION OF THE PREFERRED EMBODIMENTS

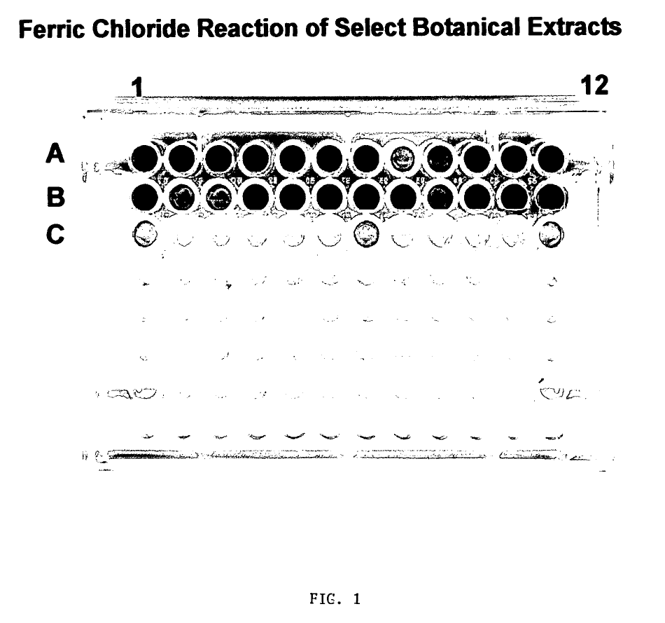

The present invention will now be described with reference to FIGS. 1-16 of the drawings.

Method of Extraction:

Botanical extracts were prepared as follows: Approximately 10 grams of leaves, berries, seeds, or tree catkins, were blended with 90 grams of water or 90 grams of 50% alcohol (ethanol) in a blender for 2 minutes (“Liquify” setting) at room temperature. The blended material was centrifuged for 10 minutes at 3,000 rpm at 5 degrees C., and the clarified supernatant harvested by decantation into sterile glass flasks. The clarified extract was stored at 4 degrees C. for further study. In the case of commercial powders, 10 grams of powders were weighed out dissolved directly into 100 mL of distilled water or 50% ethanol. Polyphenol Test: Assay conditions Fifty microliters (50 uL) of each botanical extract was deposited in one well of a 96-well culture dish (see FIG. 1) and reacted with 50 uL of an aqueous solution of ferric chloride (10%) deposited in the same well. An instantaneous color change occurs giving a green-black color of varying intensity correlated with the amount of polyphenols present in the extract. As a control for color change 100 uL of un-reacted Ferric Chloride was deposited in wells C1, C7 and C12. All extracts were tested for their polyphenolic content by reacting them against a 10% aqueous Ferric Chloride solution. Polyphenols will react with Ferric Chloride (oxidant) and turn from an orange color to a dark green to black color if the botanical polyphenol content has strong antioxidant activity. The antioxidant activity of botanical polyphenols was measured calorimetrically by the intensity of green-black reaction product formed in the test.

TABLE 1
Botanical Extracts Surveyed by Ferric Chloride Reaction
Row A:

| | |
|---|---|
| 1. | Corn Tassel extract in 50% ethanol (2× concentrated) (+++) |
| 2. | Green Tea Leaf extract (5%) in 50% ethanol (++++) |
| 3. | Pericarpium zanthoxyli (2%)-water-soluble extract (++) |
| 4. | Hydrox powder (5%) in 50% ethanol (0/+) |
| 5. | Autumn Olive berry extract (Prep II) in 50% ethanol -hexane |
| | fraction- (0) |
| 6. | Autumn Olive berry (Prep I) in 50% ethanol extract (++) |
| 7. | Corn Tassel-70% ethanol extract (+/++) |
| 8. | Powdered Tassel pellet of 70% ethanol extract (+) |
| 9. | White Willow bark (5%) extract in 50% ethanol (+++) |
| 10. | ApplepehonSH extract (5%) in 50% ethanol (+++/++++) |
| 11. | Cocoa powder extract (5%) in 50% ethanol (++) |
| 12. | Corn Tassel extract (7%) in 50% ethanol ((+++) |

Row B:

| | |
|---|---|
| 1. | Caffeic acid (1%) in 70% ethanol ((+++) |
| 2. | Gallic acid (1%) in 70% ethanol (+++) |
| 3. | Cinnamon bark powder (5%) in 50% ethanol (++) |
| 4. | Tomato paste (5%) extract in 50% ethanol (0/+) |
| 5. | Lavender dried flower extract (5%) in 50% ethanol (+/++) |
| 6. | Tilia tree dried catkin (5%) in 50% ethanol (+++) |
| 7. | Corn Tassel (7%) in 70% ethanol (+/++) |
| 8. | Autumn Olive berry (Prep II) in 50% ethanol (+/++) |
| 9. | Curly Dock dried tassel extract (10%) in 50% ethanol (++) |
| 10. | Red Pepper fresh vegetable extract in 50% ethanol (0) |
| 11. | Red Grapes fresh fruit extract in 50% ethanol (0/+) |
| 12. | Baes dried pepper korn spice extract in 50% ethanol (++/+++) |

Row C:
Numbers: 1, 7 and 12 are wells with 50 uL of un-reacted 10%
aqueous Ferric Chloride solution
Note:
positive signs (+, ++, +++) denote increasing strength of Ferric Chloride reaction as measured by a colorimetric score; (0), denotes no reaction.

Polyphenolic Content of Botanicals:

Table 1 shows the botanical extracts prepared. FIG. 1 shows results from 24 different botanical test substances. The most reactive botanicals are: 1) a proanthocyanidin-rich extract prepared from unripe apples and available commercially (Applephenon SH®, FIG. 1, A10) (obtained from Nikki Whiskey Distilling Co, Ashai Breweries, Ltd, Tokyo, Japan, 2) a hydroxytyrosol-enriched extract derived from hydrolyzed aqueous olive pulp (HIDROX™, obtained from CreAgri, Inc, Hayward, Calif.), FIG. 1, A4), 3) a hydroalcoholic extracts of Autumn Olive (Eleagnus umbelifera) ripe berries, (FIG. 1, A6), 4) water and hydroalcoholic extracts from green tea leaves (Camellia sinensis), (FIG. 1 A2), 5) White Willow bark extract enriched in polyphenols (Active Concepts, Middlesex, N.J.), (FIG. 1, A9), 6) a hydroalcoholic extract of Linden (Tilia) tree catkins, (FIG. 1, B6), 7) a hydroalcolholic extract of seeds of Curly Dock, (FIG. 1, B9), 8) water extracts of the dried fruit of prickly ash (Pericarpium zanthoxylli), (FIG. 1, A3).

In addition, the following botanical extracts were found to be positive in the Ferric Chloride test: a water extract of powdered corn tassels (PTE), hydroalcoholic extracts of dried herb leaves of Oregano, Hops, Lavender flowers, dried “Baes” seeds, Cocoa powder, Cinnamon bark, and dried catkins of the Oak tree. By contrast no antibacterial activity was found for the water-soluble extract of Anise seeds, while the 50% hydroalcoholic extract of Anise did display antibacterial activity. In support of these results, a diphenylpicrylhydrazine (DPPH) assays were conducted (data not shown), which confirmed the antioxidant activity of all of the above extracts.

Microbiological Tests:

Three different microbiological assays were employed 1) self-sterilization test, 2) challenge test, 3) post-challenge test.

1). Self-Sterilization Assay:

In the self-sterilization assay, the extracts were inoculated directly into bacterial culture medium to ascertain if there were any bacteria that would grow out upon incubation at 37 degrees C. A negative response would indicate that the extract has endogenous bactericidal activity. The extracts were assessed for self-sterilization by inoculating small aliquots of a 10% (w/v) botanical extract into sterile BHI medium and incubated at 37 degrees C. for 48 hours. Self-sterility was shown to be a property of extracts from Autumn olive berry, ApplephenonSH, green tea, prickly ash, dried curly dock flowers, dried Oregano leaves, dried Hops leaves, dried Lavender flowers, dried Baes “pepper korns”, Cocoa powder, Cinnamon bark, and Linden tree catkins. If the self-sterility test was negative, a second microbiological test was employed, called the challenge assay.

2). Challenge Assay:

In the challenge assay, various dilutions of each self-sterilizing extract were challenged by the adding a 10 uL aliquot containing 1×10⁵ S. epidermis bacteria to 1.0 mL of sterile saline and also continuing various concentrations of the test botanical extracts. The challenge assay tubes were incubated at 37 degrees C. for 48 hours. The assay included a positive control in which no botanical extract is added to the test. Positive controls become slightly turbid indicating the growth of the bacteria, while assays containing botanical extracts remain clear, indicating the bactericidal activity.

3). Post-Challenge-Test

To show that the botanical extracts had actually killed the bacteria rather merely preventing their growth, small aliquots of the challenge media were further re-inoculated into fresh sterile BHI bacterial culture medium for an additional 48 hours. Bacterial growth is expected from the positive control tubes, while the absence of any bacterial growth in the post-challenge botanical extract tests assures that the extract has killed all 10⁵ bacteria that were initially added in the challenge assay.

### EXAMPLE 1

### Microbiological Results

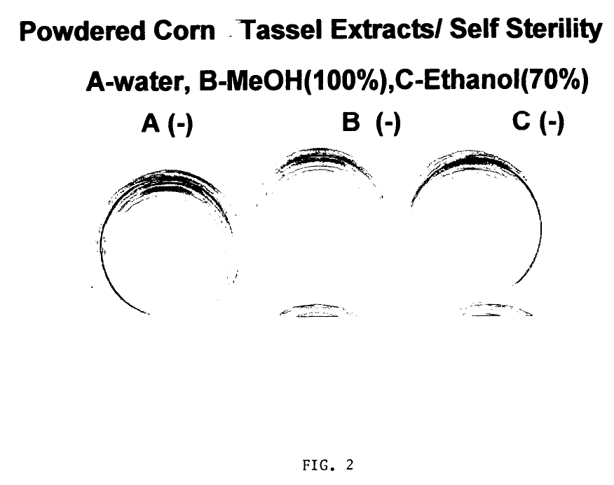

FIG. 2 is a photograph showing typical results of self-sterilization assays for powdered corn tassel extract (Tasselin). The extract was tested for sterility as a water extract (A), and methanol (B) and ethanol (C) extracts produced by sequential extraction of the water extract by methanol followed by ethanol. The three dishes labeled A, B, and C clearly indicates that no endogenous bacteria in the extracts survived the action of the antibacterial agents in Tasselin. The positive dish which had heavy bacterial growth is not shown.

Challenge test data photographs are not presented here because they do not contain a sufficient amount of bacterial growth medium to allow detectible growth, and as explained above, the absence of growth in the challenge test may only be due to a bacteriostatic effect making it necessary to examine the results of the post-challenge test to truly determine that the challenge with a given dilution of the botanical extract killed all of the spiked S. epidermis present in the challenge test. With this caveat in mind, only the results of the post-challenge assays are presented below.

### EXAMPLE 2

### Autumn Olive Berry Extract

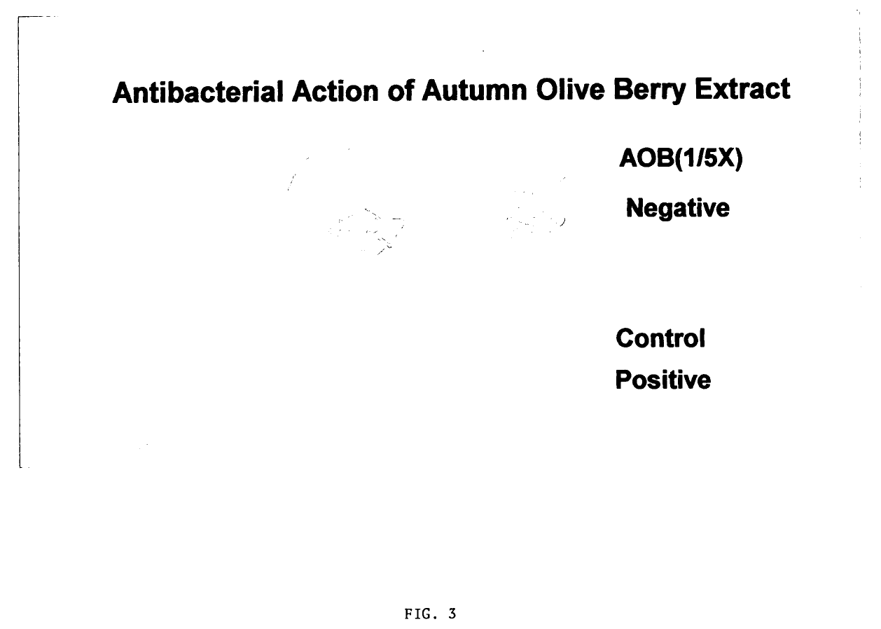

FIG. 3 present a photograph of duplicate culture dishes after post-Challenge-testing, bearing a ⅕× dilution of a hydroalcoholic extract (10% ethanol) of Autumn Olive ripe berries (top row). There was a 7-log killing of S. epidermis. The positive control also containing 10% ethanol (bottom row) shows that in the absence of the botanical extract the bacteria grew in the BHI culture medium. This is seen as a darker plate color.

### EXAMPLE 3

### Hydroxytyrosol (HIDROX® Versus Apple Extract (ApplephenonSH®)

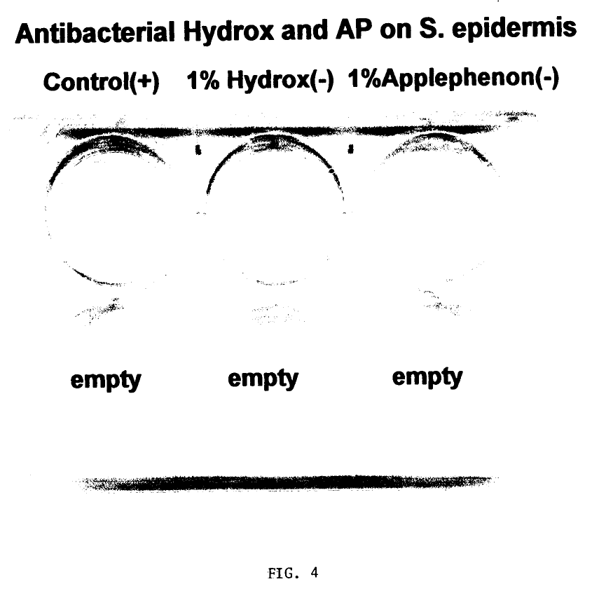

FIG. 4 compares HIDROX®, a commercial preparation of water-soluble olive pulp extract enriched in the antioxidant hydroxytyrosol for its antibacterial activity at 1% (w/v) compared with a commercial preparation of an green apple extract (ApplephenonSH®) at 1% (w/v) that is enriched in antioxidant polyphenols and catechins. Both were effective in fully killing all of the spiked S. epidermis bacteria (top middle and right wells). Control positive saline wells which were spiked with S. epidermis was contaminated with bacteria (top left well).

### EXAMPLE 4

### Green Tea Leaf Extract

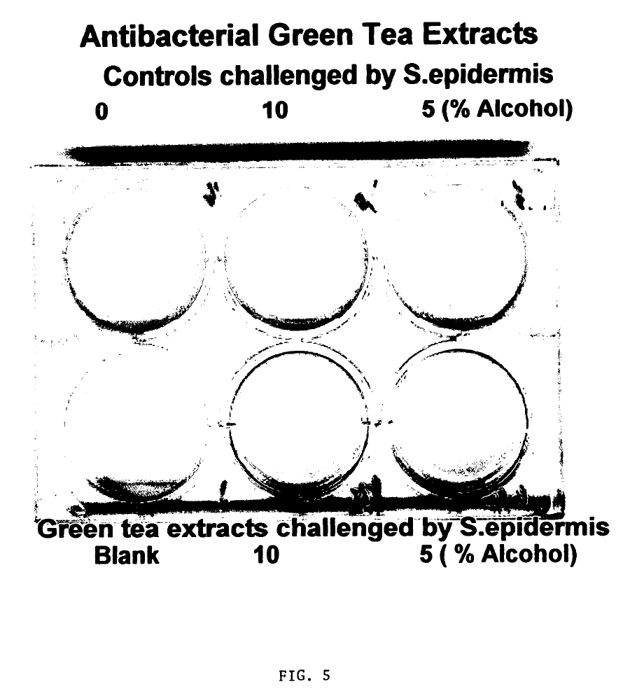

FIG. 5 present results showing the lack of antibacterial activity after post-challenge testing of green tea extracts against S. epidermis bacteria. The top three wells show that without the green tea extract the challenge test with S. epidermis grew heavy bacteria (dark colored wells) even in the presence of 5% and 10% ethanol. The bottom three wells show that green tea hydroalcoholic extract at both 5% and 10% ethanol was effective in the post challenge assay in killing five-logs of S. epidermis bacteria. These results show that the green tea leaves contain active antibacterial agent(s) related to their various polyphenolic contents.

### EXAMPLE 5

### Prickly Ash Water-Soluble Extract

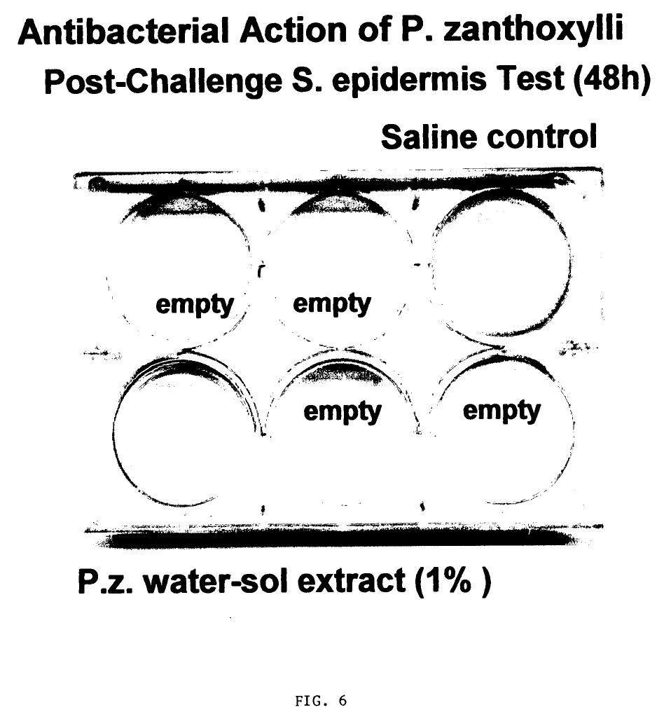

FIG. 6 presents a photograph of a 6-well culture dish, after post-challenge testing showing lack of antibacterial activity of a 1% of water-soluble extract of Prickly Ash. The saline control grew heavy S. epidermis bacteria in sterile BHI culture medium (top right well), while no bacteria grew in the 1% Prickly Ash extract (bottom left well).

### EXAMPLE 6

### Corn Tassel Powder Water-Soluble Extract (Tasselin)

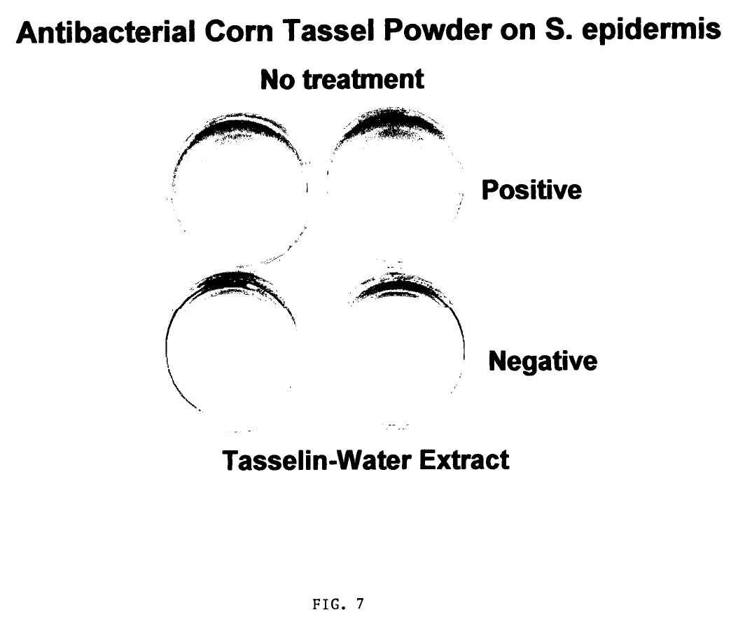

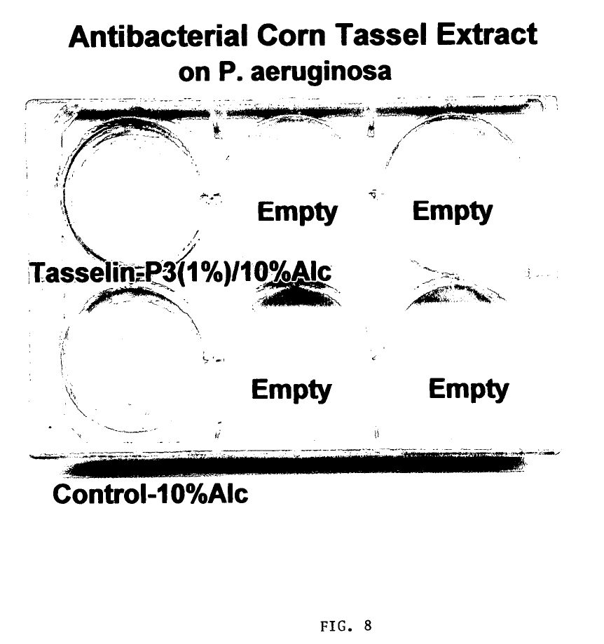

FIG. 7 presents a photograph of four (4) 35 mm disposable culture dishes after post-challenge testing, showing there was luxuriant bacterial growth in the duplicate non-treatment dishes, and no growth occurred in the duplicate dishes containing the water-soluble corn tassel extract. FIG. 8 presents results showing that a hydroalcoholic extract of corn tassels (Tasselin) is bactericidal at 1% against the gram-negative Pseudomonas aeruginosa bacteria.

### EXAMPLE 7

### Allspice (Pimenta) Extracts

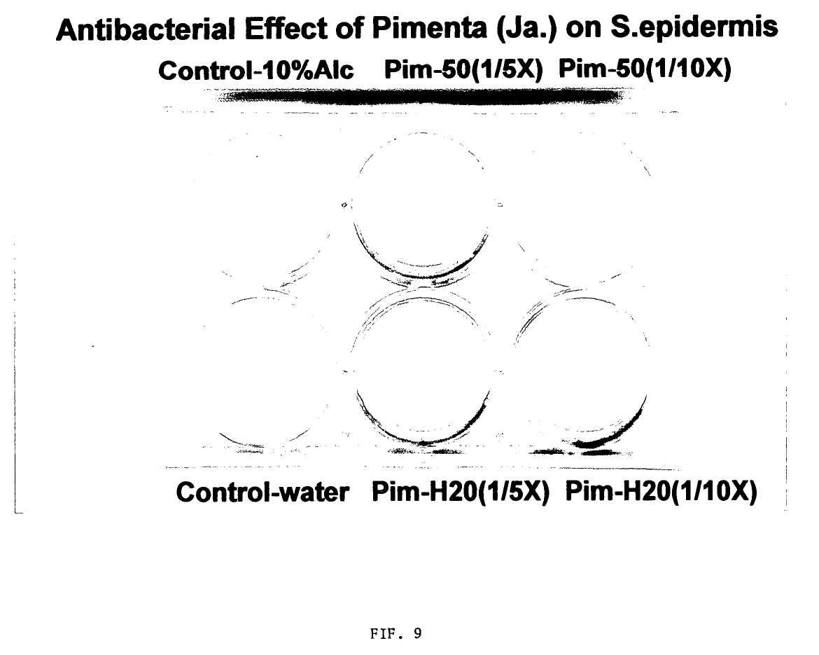

FIG. 9 presents a photograph of six (6) 35 mm culture dishes after post-challenge testing, showing that in both the 10% ethanol (top left) and water control (bottom left) wells there was luxuriant bacterial growth, while both the water-soluble (bottom middle) and hydroalcoholic extracts of Allspice (Pimenta) there was no growth at 20% of full extract strength. There was no bacterial growth in the 10% of full strength water extract, but the 10% of full strength hydro-alcoholic extract was not sufficiently potent to kill all of the bacteria.

### EXAMPLE 8

### Extracts of Oregano leaves, Oak tree catkins, and Hops leaves

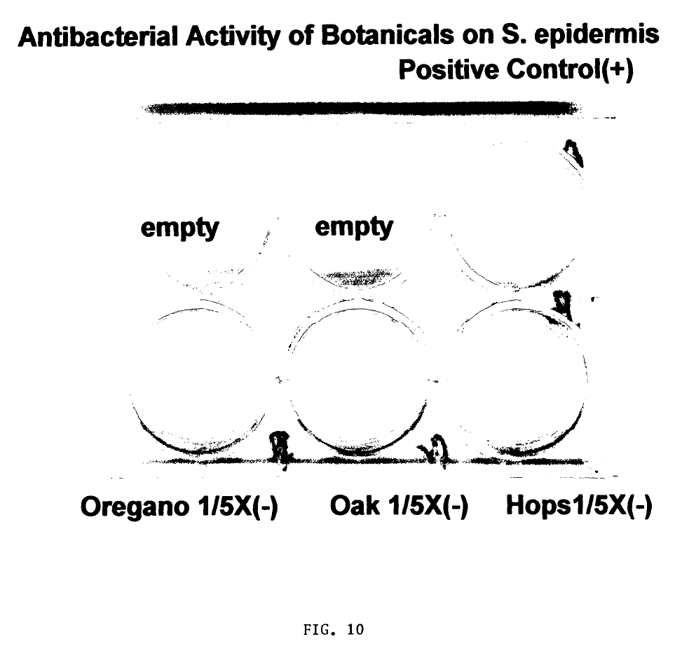

FIG. 10 presents a photograph of a 6-well culture dish after post-challenge testing showing the lack of bactericidal activity of 20% of full strength hydro-alcoholic extracts of Oregano leaves (bottom left), Oak (Quercus) dried catkins, and dried Hops flowers. A saline control (top right), with no botanical extract was positive for luxuriant bacterial growth.

### EXAMPLE 9

### Extracts of Curly Dock, Lavender and Baes “pepper korns”

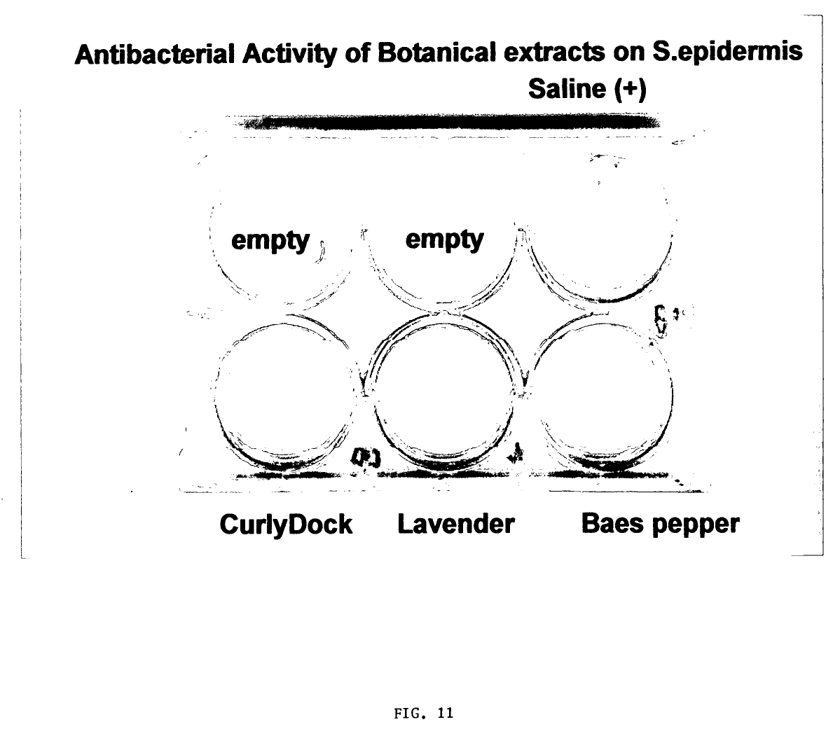

FIG. 11 presents a photograph of a 6-well culture dish after post-challenge testing, showing the lack of bactericidal activity of 20% of full strength hydro-alcoholic extracts of Curly Dock (bottom left), dried Lavender flowers, (bottom middle), and dried Baes pepper korns (bottom right). Bacterial growth occurred in the saline control, without any botanical extract (top right).

### EXAMPLE 10

### Minimal Bactericidal Concentration of ApplephenonSH®

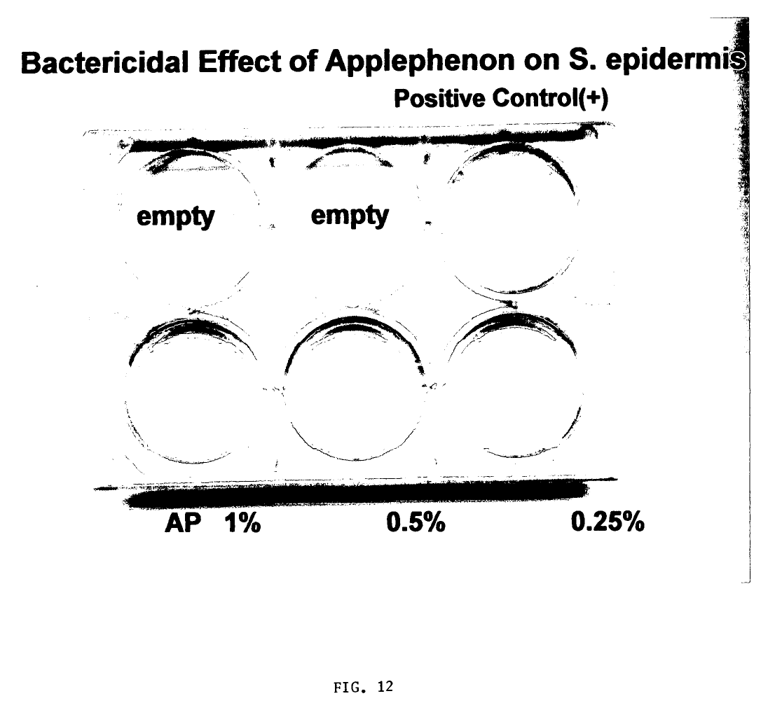

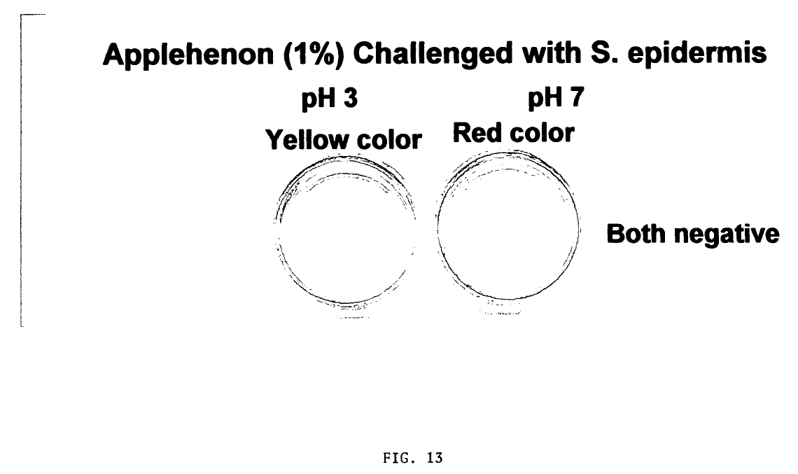

FIG. 12 presents a photograph of a 6-well culture dish after post-challenge testing, showing in the top (right well) that bacteria grew in the sterile BHI medium (dark color), while a 1% and 0.5% and 0.25% water solution of ApplephenonSH(& (bottom three wells) prevented bacterial growth as evidenced by clear BHI medium. The minimal bactericidal concentration of ApplephenonSH® was determined to be about 0.063%. An independent study (FIG. 13) showed that equivalent bactericidal activity was present in ApplephenonSH extracts that were prepared in water at pH 3.5 and at pH 7.0. This dispels that the color change to red and the neutral pH alter the bactericidal activity of ApplephenonSH®.

### EXAMPLE 11

### Antifungal(Yeast) Activity of ApplephenonSH®

In another independent study the yeast, A strain of commercial bakers yeast (Saccharomyces) was cultured in a balanced salt solution of glucose (0.45%) in DMEM medium (BioWhittaker/CAMBREX, Walkersville, Md.). ApplephenonSH® was added at 1%. It killed 9-logs of these yeast cells in the challenge assay.

### EXAMPLE 12

### ApplephenonSH is a Broad-Acting Bactericide

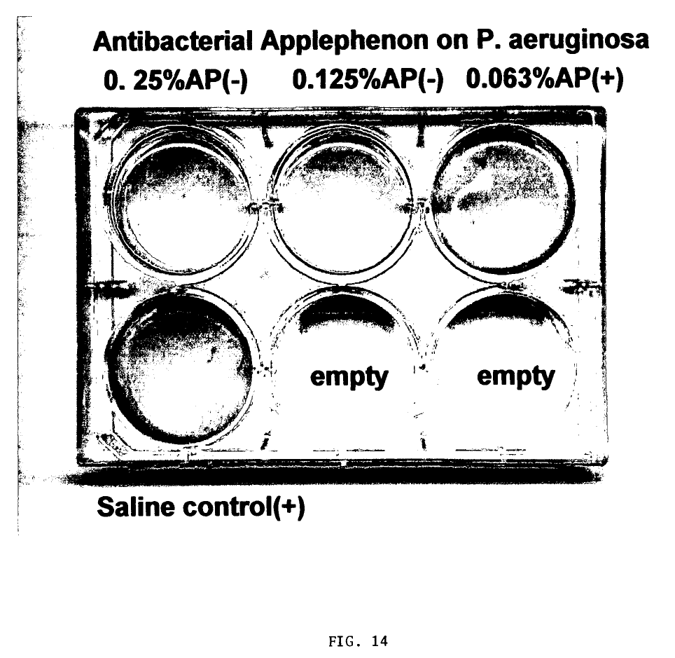

FIG. 14 present results showing that ApplephenonSH® is bactericidal for the gram-negative bacterium, Pseudomonas aeruginosa. It shows that the minimal bactericidal concentration for ApplepenonSH® is about 0.125%. A broad-acting bactericidal action was also displayed against a number of non-motile and motile rod-shaped bacteria present in isolates from human underarm skin.

### EXAMPLE 13

### Whole Clove Extracts and Anise Seed Extracts

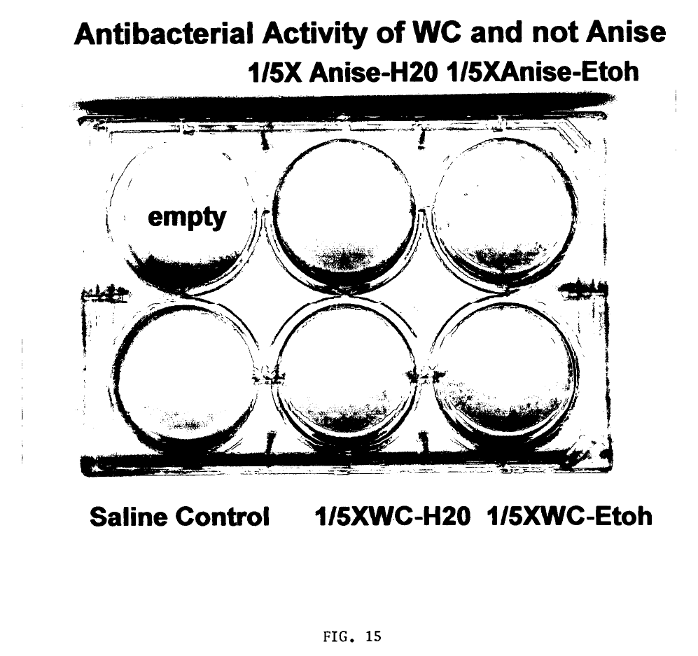

FIG. 15 shows the bactericidal activity of whole cloves (WC) and Anise at 20% (⅕×) of full-strength of extracts prepared either in water (H₂O) or in 50% ethanol (ETOH). The word “empty” denotes the absence of any tests in those wells. The saline control (bottom left well), and the 20% (⅕×) Anise-H₂O both spiked with S. epidermis were heavily contaminated with spiked bacteria.

### EXAMPLE 14

### Combinations of Antimicrobial Botanicals

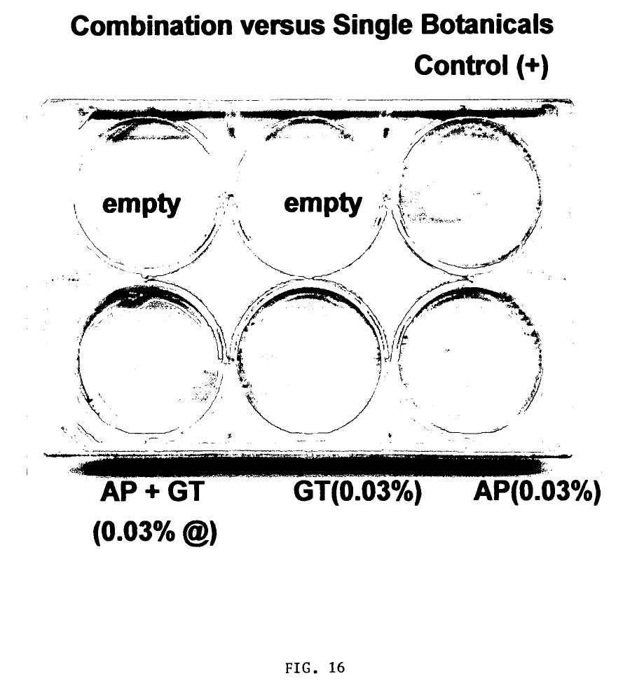

Pair-wise combinations were made of some of the antibacterial botanicals disclosed in this invention. FIG. 16 presents results showing that ApplephenonSH® extract (AP) at 0.03% (right bottom well) or Green tea extract (GT) at 0.03% ((middle bottom well) were individually ineffective in inhibiting P. aeruginosa bacterial growth (dark color like that seen in the positive control, top right well). By contrast, the combination (GT+AP, left bottom, light colored well)), adjusted to a combined concentration of 0.03%, was effective in this pair-wise combination in killing 7-logs of this gram-negative bacterium. The other pair-wise results indicate that ApplephenonSH® can enhance the bactericidal activity of prickly ask and corn tassel botanical extracts.

### EXAMPLE 15

### A Typical Formulation Using Select Antimicrobial Botanical Extract

Example 15 shows a typical cosmetic formulation in which an aqueous extract of powdered ApplephenonSH® is the antibacterial botanical agent that was formulated in a oil droplet encapsulated starch matrix oil-in-water emulsion, (ThixoDerm,(INCI name).

### Example 15

### An antibacterial lotion containing ApplephenonSH®:AppleDerm

| | |
|---|---|
| Component | Amount (%) |

A. Aqueous Phase

| | |
|---|---|
| Natural Corn Starch | 3.3 |
| Glycerol | 5.0 |
| Varisoft AT 100 | 2.4 |
| ApplephenonSH ® | 0.1 |
| Distilled Water | 73.5 |

B. Oil Phase

| | |
|---|---|
| Coconut Oil: (70:30) | 9.3 |
| Dimethicone (Fluid DC-200)* | 0.8 |
| Poly-cyclopentanesiloxane (Fluid DC-245) | 3.2 |
| Citricidal ® | 0.5 |

There has thus been shown and described novel anti-microbial botanical extracts and methods for making and testing the same, of which fulfill all the objects and advantages sought therefore. Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to be limited only by the claims which follow.

I claim:

1. A method for the preparation of antimicrobial botanical actives comprising the steps of:
(a) selecting a plant from the group consisting of: Allspice (Pimenta diocia), Applephenon®, a commercially available green apple extract (Nikka Whisky Distillery, Ashai Breweries Ltd, Tokyo, JP), ripe autumn olive berry (Eleagnus umbelifera), dried “Baes pepper korns from the Island of Guadalupe, commercially available cinnamon powder, commercially available cocoa powder, cloves (Syzgium aromaticum), corn tassels (Zea mays), curly dock seeds (Rumex crispus), dried hops leaves (Humulus lupulus), dried Lavender flowers (Lavendula provence), Linden tree catkin (Tilia americana), dried Oak tree catkins (Quercus alba), dried Oregano leaves (Origanum vulgare), chinese Prickly Ash (Pericarpium zanthoxylli), and commercially available dried green tea leaves (Camellia sinensis); and
(b) forming a natural botanical extract from said plant in an amount effective for exerting at least one of the following properties and physiological actions on microbes:
(1) inhibiting the growth of gram-positive and gram-negative microorganisms, and
(2) inhibiting the growth of yeast and fungi.

2. A method according to claim 1, wherein the step of forming an extract comprises the steps of:
(1) blending at least one of berries, seeds and tree catkins of the plant with at least one of water and ethanol to prepare a blended material; and
(2) subjecting the blended material to a centrifuge to prepare a clarified supernatant.

3. A method according to claim 2, further comprising the step of decanting the supernatant into a sterile container and storing the sterile container with the clarified supernatant at approximately 4 degrees C.

4. A method according to claim 2, wherein the blending step is carried out at room temperature and the blended material is centrifuged at approximately 5 degrees C.

5. A method for determination of the anti-microbial activity of the natural botanical extract prepared according to claim 1, using at least one of detailed self-sterilization, challenge and post-challenge tests.

6. A method for the determination of the antioxidant and or polyphenolic content of the natural botanical extract prepared according to claim 1, using about a 10% aqueous Ferric Chloride solution.

7. A method for the measuring the antioxidant activity of the natural botanical extract prepared according to claim 1, as determined by a spectroscopic analysis using a 2.5% dilution of a 25 ug/ml stock solution of diphenylpicyrl hydrazine.

8. The topical application to a mammal of the natural botanical extract prepared according to claim 1 for the treatment of infectious skin conditions and malodor in antiperspirant and anti-odor personal care and skin care formulations to eliminate microbial growth.

9. The topical application to the skin of a mammal of the natural botanical extract prepared according to claim 1 to eliminate microbes and thereby malodors in chronic wounds.

10. Use of the natural botanical extract prepared according to claim 1 as an ingredient in formulations for skin care, personal care and cosmetic products.
